(19) United States
(12) Patent Application Publication
(10) Pub. No.: US 2024/0249830 A1
(43) Pub. Date: Jul. 25, 2024

(54) SYSTEMS AND METHODS FOR MANAGING PATIENT CARE INFORMATION AND COMMUNICATIONS ACROSS A PLURALITY OF SERVICE PROVIDERS

(71) Applicant: Vitare Health, San Diego, CA (US)

(72) Inventors: Franklin Moses, San Diego, CA (US); Gavin Russell Wiswell, San Marcos, CA (US); Sean Joseph Chapman, Escondido, CA (US); Devin Marie Clark, Murrieta, CA (US); Mark Edward Hansen, Redlands, CA (US); Kristoher Reichert, Washington, DC (US)

(21) Appl. No.: 18/420,720

(22) Filed: Jan. 23, 2024

Related U.S. Application Data
(60) Provisional application No. 63/440,828, filed on Jan. 24, 2023.

(51) Int. Cl. G16H 40/20; G16H 10/60

(52) U.S. Cl. CPC G16H 40/20; G16H 10/60

(57) ABSTRACT

Aspects of the present disclosure provide systems, methods, apparatus, and computer-readable storage media that provide for a system that is able to implement improved coordination of data among one or more computing systems, generally owned and controlled by different parties, in order to facilitate a system that provides “patient-centric” care and improves overall patient outcomes. In one example case, a user interface dashboard may be generated that efficiently displays the compiled data from various sources, whereupon systems may determine and display action items for a user of the dashboard to implement according to pre-determined rules of the systems. The dashboard may provide multiple views to a user to allow for efficient handling of specific tasks as determined by the system.

### CROSS-REFERENCE TO RELATED APPLICATIONS

This application claims the benefit of priority from U.S. Provisional Application No. 63/440,828, filed Jan. 24, 2023 and entitled “SYSTEMS AND METHODS FOR MANAGING PATIENT CARE INFORMATION AND COMMUNICATIONS ACROSS A PLURALITY OF SERVICE PROVIDERS,” the disclosure of which is incorporated by reference herein in its entirety.

### BACKGROUND

Technology systems play a critical role in the health care field. In fact, different technological systems are utilized in almost every type of health care setting to, for example, manage patient workflow, handle patient billing, monitor resource utilization (rooms, tools, supplies, personnel), etc. Many of these systems are limited to special use cases at a particular provider location and are designed specifically for each provider and task. For example, a hospital may have a system that tracks room usage, staffing resources, and various tasks that need to be completed for a patient (e.g., follow up, tests, labs, etc.). This system may also be part of, or tied into, a billing system that can specifically track and monitor patient care to determine the codes for which that hospital can bill. Conversely, physicians' offices track very different aspects of patient care and are not required to monitor the usage of similar resources. Still further, medical device equipment providers have their own systems to monitor equipment use. These providers need to keep track of logistics for delivery, the types of supplies that are needed, ensure that resupply times are accurate as needed, and the like. Their billing systems also have other considerations to manage that are very different than a hospital or physician's office. Moreover, pharmacies use separate systems used to track patient medications, refill timing, and to manage communications between providers and customers.

While each of these technological systems have contributed to better patient care and outcomes, one problem with current systems is that they are generally siloed within their various operating environments. This is an understandable situation as each technological system would be specifically designed for an end use customer purchasing the system. Accordingly, while these systems are designed to be helpful to a specific entity providing patient care to a patient within a specific circumstance (e.g., while undergoing a hospital stay), they are not necessarily designed to maximize patient outcomes when taking into account the needs of a patient as a whole.

Efforts have been made to provide for some cross-compatibility for data between systems in a few areas. For example, medical records and medical imaging data often utilize standardized formatting to allow users at different systems to access data. These efforts at least allow a physician in an office to view, e.g., medical images or lab results taken at a different location in order to manage further care. However, these technological systems fall dramatically short in addressing the multi-faceted areas of needs for a patient. This is in part because there is minimal incentive for disparate providers to build out such a complicated system.

### BRIEF DESCRIPTION OF THE DRAWINGS

For a more complete understanding of the present disclosure, reference is now made to the following descriptions taken in conjunction with the accompanying drawings, in which:

FIG. 1 is a block diagram of an example of a system that supports coordinated patient care according to one or more aspects;

FIG. 2 shows a screenshot of a coordinated care system dashboard according to one or more aspects;

FIG. 3 shows a screenshot of a coordinated care system dashboard according to one or more aspects;

FIG. 4 shows a screenshot of a coordinated care system dashboard according to one or more aspects;

FIG. 5 shows a screenshot of a coordinated care system dashboard according to one or more aspects;

FIG. 6 shows a screenshot of a coordinated care system dashboard according to one or more aspects;

FIG. 7 shows a screenshot of a coordinated care system dashboard according to one or more aspects;

FIG. 8 shows a screenshot of a coordinated care system dashboard according to one or more aspects;

FIG. 9 shows a screenshot of a coordinated care system dashboard according to one or more aspects;

FIG. 10 shows a screenshot of a coordinated care system dashboard according to one or more aspects;

FIG. 11 shows a screenshot of a coordinated care system dashboard according to one or more aspects;

FIG. 12 shows a screenshot of a coordinated care system dashboard according to one or more aspects;

FIG. 13 is an example system architecture that facilitates a coordinated patient care system according to one or more aspects;

FIG. 14 is a flow diagram illustrating a method in accordance with one or more aspects; and

FIG. 15 is a flow diagram illustrating a method in accordance with one or more aspects.

It should be understood that the drawings are not necessarily to scale and that the disclosed aspects are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular aspects illustrated herein.

### DETAILED DESCRIPTION

Aspects of the present disclosure provide systems, methods, apparatus, and computer-readable storage media that provide for coordination of data among one or more computing systems, generally owned and controlled by different parties, in order to facilitate a system that provides “patient-centric” care and improves overall patient outcomes. The following discussion provides illustrative concepts which accentuate contrasting comparisons between a normal patient protocol by multiple care providers, compared to a “patient-centric” environment provided for by aspects of the present disclosure. For example, in a normal patient protocol, the various service providers are focused on their specific relationship with the patient based on the piece of service they provide. A patient-concentric model has a wholistic patient picture as the dominant focus, rather than any given piece or part. The inventive systems described herein allows the wholistic picture to remain despite the separate involvement of multiple service providers being present in the care of the patient.

According to a specific illustration of a patient-centric model, a user/patient may enroll (or be enrolled by a third party) in the novel system. Upon enrollment, the user's relevant health data may be compiled into an integrated data set from one or more sources, e.g., a hospital, medical equipment device provider, pharmacy, and the like. Compilation of this data allows for a patient centered focus of attention. With this data, aspects may then, for example, coordinate patient care, schedule/implement physician appointments, follow up on patient compliance on a treatment course, ensure supplies and prescriptions are adequately provided, implement required remote patient monitoring, and provide feedback to relevant third party systems. Aspects may further automate billing processes when requirements have been fulfilled. Additionally, aspects may notify third parties when milestones have been met for purposes of meeting third party patient care and billing requirements. Moreover, aspects may further coordinate actions that may be seen as more traditionally non-medical, but still very important for a positive outcome for a patient such as mental health checks and coordination of services as well as coordination of social services.

In accordance with another aspect of the present disclosure, a dashboard may be implemented that efficiently displays the compiled data from various sources, whereupon systems may determine and display action items for a user of the dashboard to implement according to pre-determined rules of the systems. The dashboard may provide multiple views to a user, as described more in detail below, to allow for efficient handling of specific tasks as determined by the system. Using this dashboard, a user may also cause a system to retrieve or prompt a patient to provide information, such as an indication of whether medication has been taken, whether supplies have been received, to obtain data from a remote patient monitoring device and the like.

In accordance with another aspect, a patient app may be provided that implements actions to further facilitate patient care in conjunction with the described systems. The app may implement functionality to handle one or more of: enrolling a patient in the coordinated care system, providing a patient with reminders, receiving input data from a patient regarding compliance and further needs, assisting with scheduling follow up care, facilitating communication with an operator of a coordinated care system, retrieving and transmitting data from remote health monitoring devices, providing information to a patient relevant to further patient needs, etc.

The foregoing has outlined rather broadly the features and technical advantages of the present disclosure in order that the detailed description that follows may be better understood. Additional features and advantages will be described hereinafter which form the subject of the claims of the disclosure. It should be appreciated by those skilled in the art that the conception and specific aspects disclosed may be readily utilized as a basis for modifying or designing other structures for carrying out the same purposes of the present disclosure. It should also be realized by those skilled in the art that such equivalent constructions do not depart from the scope of the disclosure as set forth in the appended claims. The novel features which are disclosed herein, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

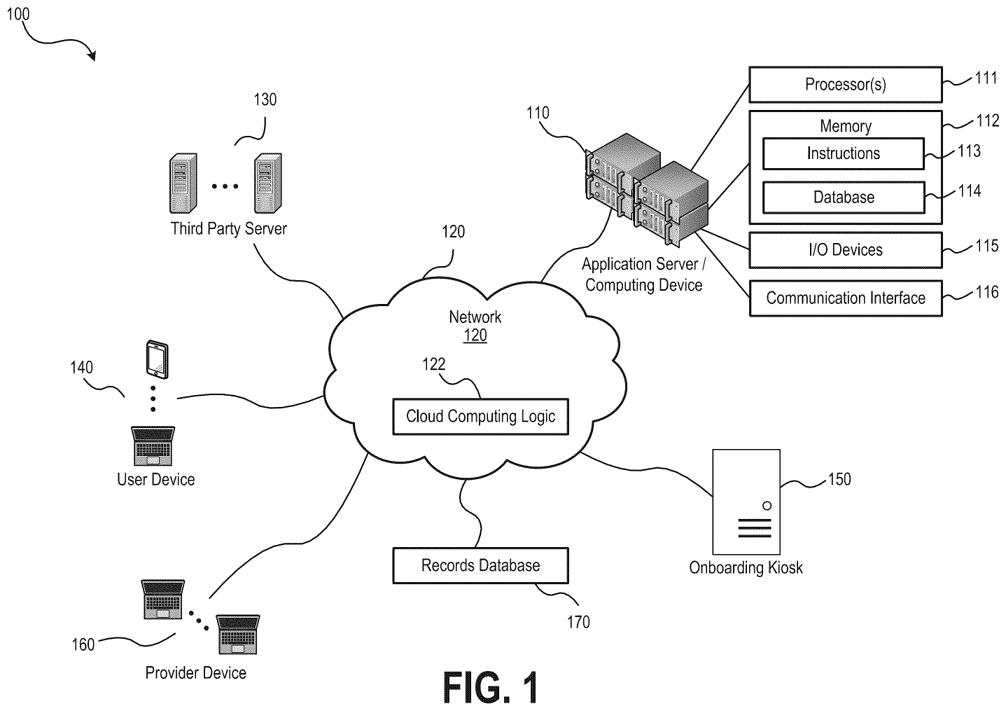

Referring to FIG. 1, a block diagram of an example of a system that supports coordinated patient care in accordance with aspects of the present disclosure is shown as a system 100. The system 100 may be configured to retrieve data for a particular patient from a plurality of sources, compute and coordinate follow up on various action items between parties involved, and provide output data based on actions taken in system 100.

As shown in FIG. 1, the system 100 includes computing device 110 communicatively coupled to third party server devices 130, user devices 140, onboarding kiosk 150, provider devices 160, records database 170, via one or more networks 120. The third party may be associated with different types of entities. Computing device 110 may implement an application server that coordinates information flow and computations within system 100. Computing device 110 may include or correspond to one or more servers, a desktop computing device, a laptop computing device, a personal computing device, a tablet computing device, a mobile device (e.g., a smart phone, a tablet, a personal digital assistant (PDA), and the like), a virtual reality (VR) device, an augmented reality (AR) device, an extended reality (XR) device, a vehicle (or a component thereof), an entertainment system, other computing devices, or a combination thereof, as non-limiting examples. The computing device 110 includes one or more processors 111, a memory 112, one or more input/output (I/O) devices 115, and one or more communication interfaces 116. In some implementations, one or more of the components may be optional or separately housed (e.g., the I/O devices 115), one or more additional components may be included in the computing device 110, or both. It is noted that functionalities described with reference to the computing device 110 are provided for purposes of illustration, rather than by way of limitation and that the exemplary functionalities described herein may be provided via other types of computing resource deployments. For example, in some implementations, computing resources and functionality described in connection with the computing device 110 may be provided in a distributed system using multiple servers or other computing devices, or in a cloud-based system using computing resources and functionality provided by a cloud-based environment that is accessible over a network, such as cloud-computing logic 122 associated with the one of the one or more networks 120. To illustrate, one or more operations described herein with reference to the computing device 110 may be performed by one or more servers or the cloud-computing logic 122 that communicates with one or more other devices.

The one or more processors 111 may include one or more microcontrollers, application specific integrated circuits (ASICs), field programmable gate arrays (FPGAs), central processing units (CPUs) having one or more processing cores, or other circuitry and logic configured to facilitate the operations of the computing device 110 in accordance with aspects of the present disclosure. The memory 112 may include random access memory (RAM) devices, read only memory (ROM) devices, erasable programmable ROM (EPROM), electrically erasable programmable ROM (EEPROM), one or more hard disk drives (HDDs), one or more solid state drives (SSDs), flash memory devices, network accessible storage (NAS) devices, or other memory devices configured to store data in a persistent or non-persistent state. Software configured to facilitate operations and functionality of the computing device 110 may be stored in the memory 112 as instructions 113 that, when executed by the one or more processors 111, cause the one or more processors 111 to perform the operations described herein with respect to the computing device 110, as described in more detail below. Additionally, the memory 112 may be configured to store data and information at one or more databases 114. In some aspects, the one or more databases 114 may store historical communications data.

In some implementations, the computing device 110 includes one or more input/output (I/O) devices 115. The I/O devices 115 may include one or more display devices, a keyboard, a stylus, one or more touchscreens, a mouse, a trackpad, a microphone, a camera, one or more speakers, haptic feedback devices, or other types of devices that enable a user to receive information from or provide information to the computing device 110. In some implementations, the computing device 110 is coupled to the display device, such as a monitor, a display (e.g., a liquid crystal display (LCD) or the like), a touch screen, a projector, a virtual reality (VR) display, an augmented reality (AR) display, an extended reality (XR) display, or the like. In some other implementations, the display device is included in or integrated in the computing device 110. The computing device 110 may also include one or more communication interfaces 116, which may be configured to communicatively couple the computing device 110 to the one or more networks 120 via wired or wireless communication links established according to one or more communication protocols or standards (e.g., an Ethernet protocol, a transmission control protocol/internet protocol (TCP/IP), an Institute of Electrical and Electronics Engineers (IEEE) 802.11 protocol, an IEEE 802.16 protocol, a 3rd Generation (3G) communication standard, a 4th Generation (4G)/long term evolution (LTE) communication standard, a 5th Generation (5G) communication standard, and the like).

It is appreciated that system 100 may have various computing resources either distributed or combined according to different aspects in order to facilitate the functionalities described herein. Additionally, various parts of system 100, e.g., third party server, 130, user device 140, onboarding kiosk 150, provider device 160, and records database 170, may be administered by a one or more of third parties and/or may include software administered by application server 110. Such modifications will be understood by a person of ordinary skill in the art in possession with the present disclosure.

Third party server 130 may include one or more servers that implement functionality and store data for one or more third party care organizations/facilities using system 100. For example, a hospital system, physician's office, medical supply provider, pharmacy location, etc., may have its own software programs for administering patient care. Patient data, billing data, orders for medications or devices, and the like, may be stored at one or more third party servers 130 corresponding to the respective provider. Such data may be received from one or more of user device 140, provider device 160 or records database 170. Further, in some aspects, application server 110 may send data back to third party server 130 and other devices of system 100 as appropriate and described herein.

User devices 140 and provider devices 160 may be computing devices including a device that corresponds to one or more servers, a desktop computing device, a laptop computing device, a personal computing device, a tablet computing device, a mobile device (e.g., a smart phone, a tablet, a personal digital assistant (PDA), and the like), a virtual reality (VR) device, an augmented reality (AR) device, an extended reality (XR) device, a vehicle (or a component thereof), an entertainment system, other computing devices, or a combination thereof, as non-limiting examples. Such devices may also include one or more processors, a memory, one or more input/output (I/O) devices, and one or more communication interfaces such as described above with respect to computing device 110.

User device 140 may be loaded with a user app that assists in communications with a user/patient and provides data to application server 110. For example, user device 140 may include a smartphone or tablet that displays one or more actions to a user and receives feedback based on actions/entries from the user. User device 140 may further include monitoring systems (or be in communication with external health monitoring systems) that take measurements such as a user's basic vitals, data relating to user sleep quality, blood glucose, heart monitoring data, feedback from user emergency monitoring (e.g., to monitor falling or other emergency situations). A user app may facilitate direct communications through a dedicated video conferencing or messaging system or may utilize native communication capabilities of device 140. Additionally, the app may access a user's calendar and scheduling functions of the device in order to, e.g., facilitate scheduling physician follow up or other care interactions.

The app may include further functionality to help a patient manage medications. For example, the app may provide reminders to a patient to take medication at appropriate times and may allow for a patient to check off a reminder when it is completed. This can assist a patient that may sometimes forget whether a medication has been taken. Additionally, the app may provide/display a picture of one or more medications that need to be taken by a patient to help the patient distinguish between various medications on hand.

Provider device 160 may be one or more devices utilized by physicians and staff for a provider entity. For example, provider device 160 may be a computer utilized by hospital staff to manage a patient stay at a hospital or at a physician's office appointment. Such a device may be utilized to enter or read results from various tests, examination data, diagnoses, physician orders and instructions, and the like. In another aspect, provider device 160 may be part of a point of sale or order entry system of a medical device provider or pharmacy. Such a system may read orders from a party treating a patient and facilitate delivery of the order as required. Provider device 160 may store such information locally or remotely at server 130 (which would generally correspond to a server handling the respective computing system). Relevant information obtained or read at device 160 may be transmitted to application server 110 from provider device 160 or server 130 over network 120.

In one aspect, records database 170 may be further utilized. Records database 170 may function in a similar manner as third party server 130 to provide information to application server 110. However, in some instances, records database 170 may further include a system that compiles relevant patient data from multiple sources. For example, if a patient has an electronic health record that compiles information from multiple service providers, such information may be sent to application server 110. This information may then be formatted into a usable format for server 110. Additionally, server 110 may also be feedback information to records database 170 to update records in light of actions taken and reported to application server 110.

In one aspect, onboarding kiosk 150 may be utilized to register a user/patient into the coordinated care system. It is appreciated that prior to patient information being provided to application server 110, requisite patient data requirements would generally need to be satisfied (e.g., obtaining personal contact information, consent to share medical information, obtain insurance/billing information, etc.). Information corresponding to such a registration would generally need to be transmitted to various providers as well prior to any information sharing. Onboarding kiosk 150 may be a computing device specifically configured for this purpose and may be implemented using, e.g., a desktop computing device, a laptop computing device, a personal computing device, a tablet computing device, a mobile device, or a combination thereof, as non-limiting examples. Such devices may also include one or more processors, a memory, one or more input/output (I/O) devices, and one or more communication interfaces such as described above with respect to computing device 110. Onboarding kiosk 150 may communicate with application server 110 over network 120, or may have more direct communication established therebetween. It is further appreciated that onboarding of a user/patient may be implemented using other aspects of system 100, such as by utilizing user device 140, or with the assistance of a provider using provider device 160. Once a user is registered in the coordinated care system to the satisfaction of the various parties involved, information flow may occur across system 100 as described herein.

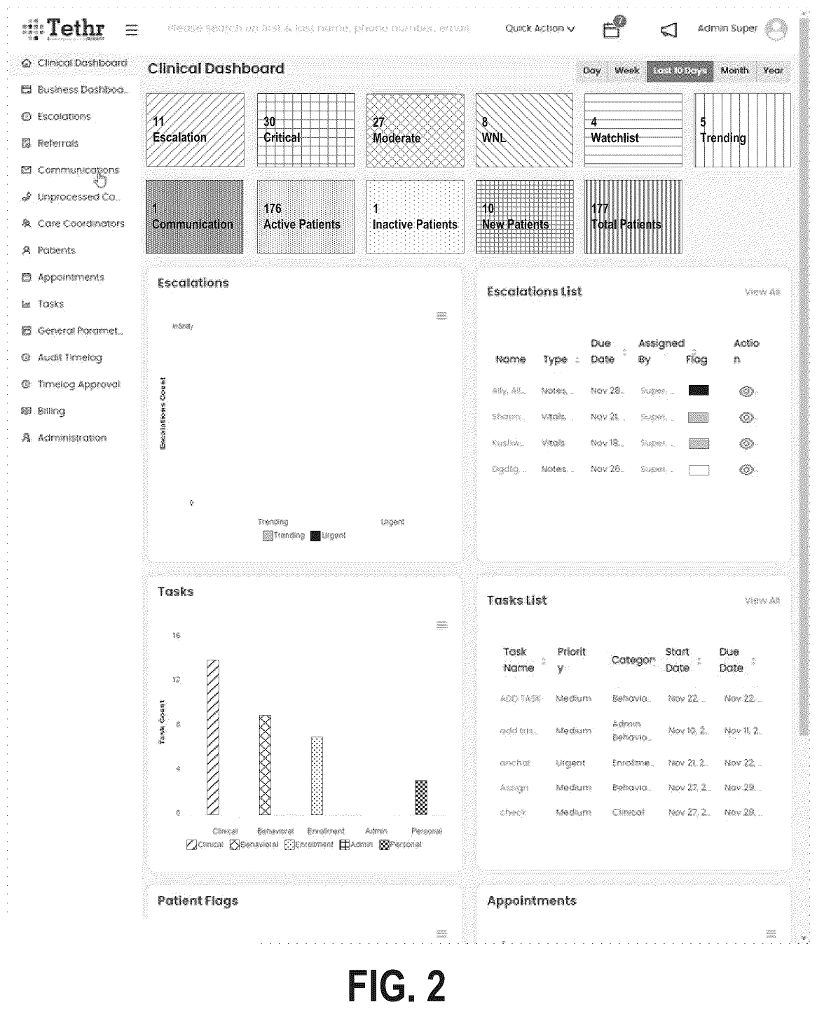

FIGS. 2-12, as well as the examples and methods discussed below, illustrate multiple aspects of the present disclosure for implementing a coordinated care system. Such aspects may be performed by one or more computing devices, such as the devices shown in system 100. The various views and functionalities are generated by and within the coordinated care system to assist a user in providing patient centric care to a patient in a manner that allows the user to see a full picture of a patient, follow up with a patient, receive feedback from the patient or one or more providers, etc. FIG. 2 is a screenshot of an example implementation of a user dashboard for a user/administrator of a coordinated care system. As shown, the dashboard includes selectable tabs (shown on the left) where a user can navigate between different screens/views, which are described in more detail below, based on the needs of the user. Color coded selectable buttons are provided (shown on top) that correspond to different portions that an administrator may want to view. For example, selecting the Escalation button will load a further screen that will list escalated tasks that the user may need to address. Selection of the Critical button may load a different set of tasks for follow up. The communications button may load a screen showing incoming communications and/or tasks involving communications that need to be completed. Other buttons may also be provided which may provide different views that may be helpful to an administrator of the system. Graphical presentations and charts may also be displayed (shown on bottom) that provide a user with alternate views of different pending states of aspects of the coordinated care ecosystem. These views may provide a user a more easily discernable picture of issues and needs that may require attention within the system such as a task list, escalation list, number of tasks by different types of category (e.g., sorted by urgency, or type of patient needs), etc.

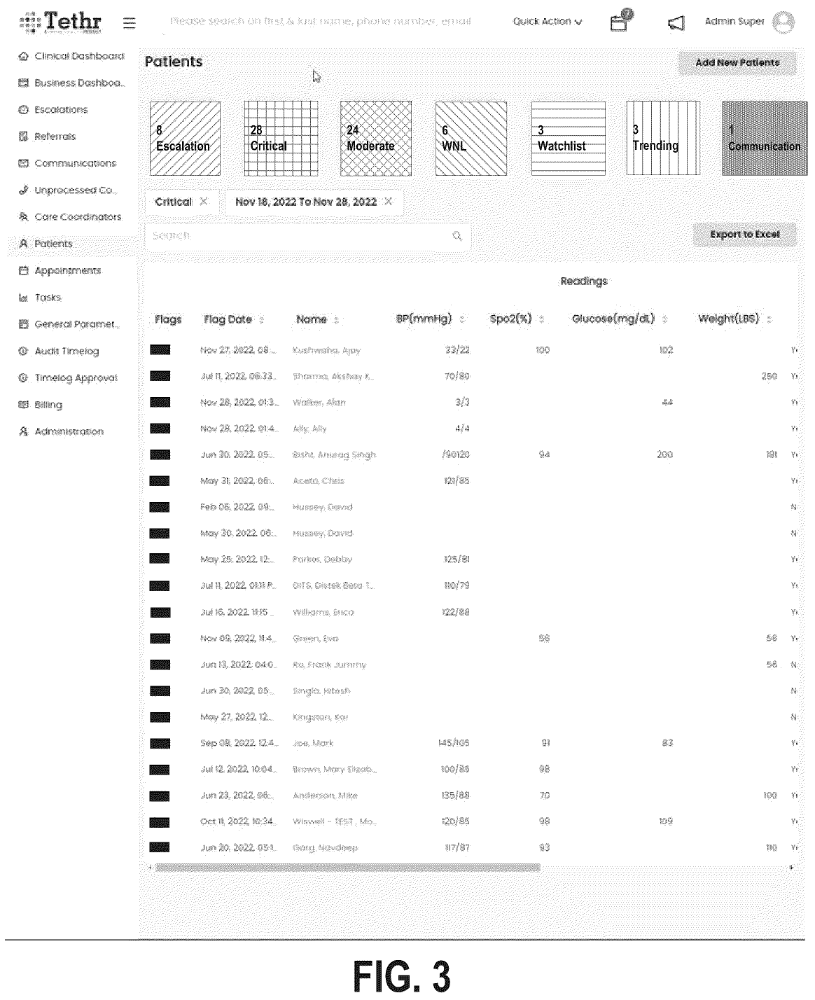

FIG. 3 is a screenshot of the system shown in FIG. 2 after the user has clicked the Critical button. As can be seen, a patient list is provided corresponding to patients with needs that are deemed critical by the system. The list includes a color coded flag, a date corresponding to the flag item, along with other relevant patient information (e.g., various vital readings). Further, the tab menu on the left now has the Patients tab selected. This tab provides an alternative route to viewing patient lists. A user navigating from that tab that would want to see the illustrated view would also select the Critical button provided at the top of this screen to sort the patient list accordingly.

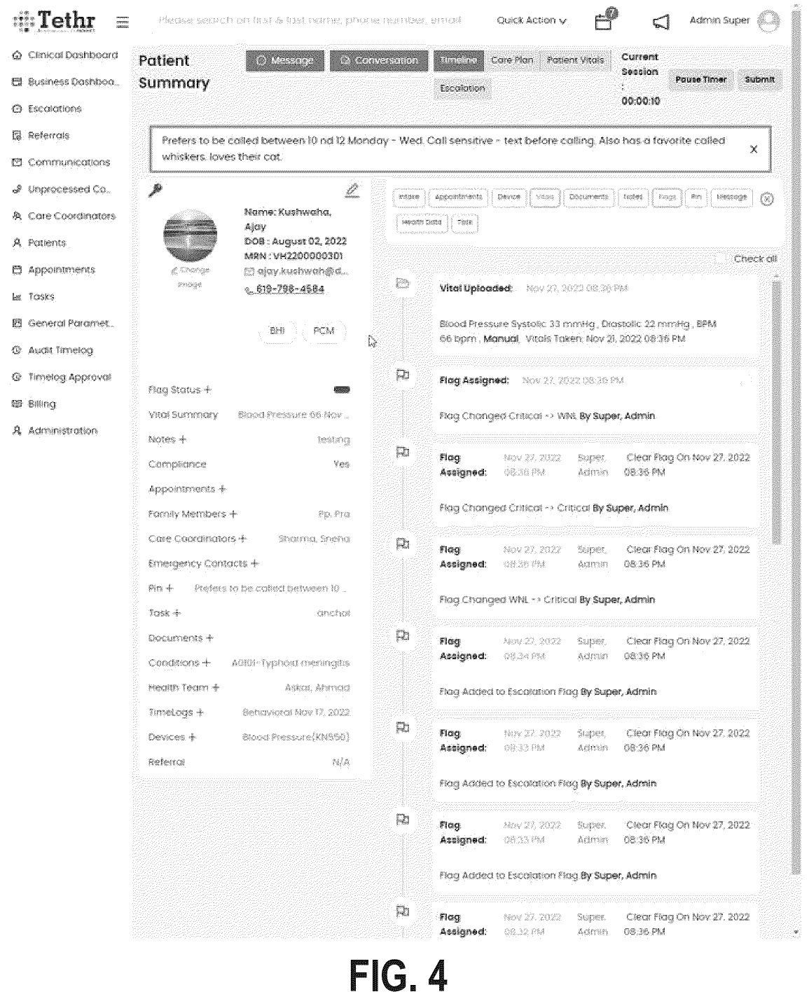

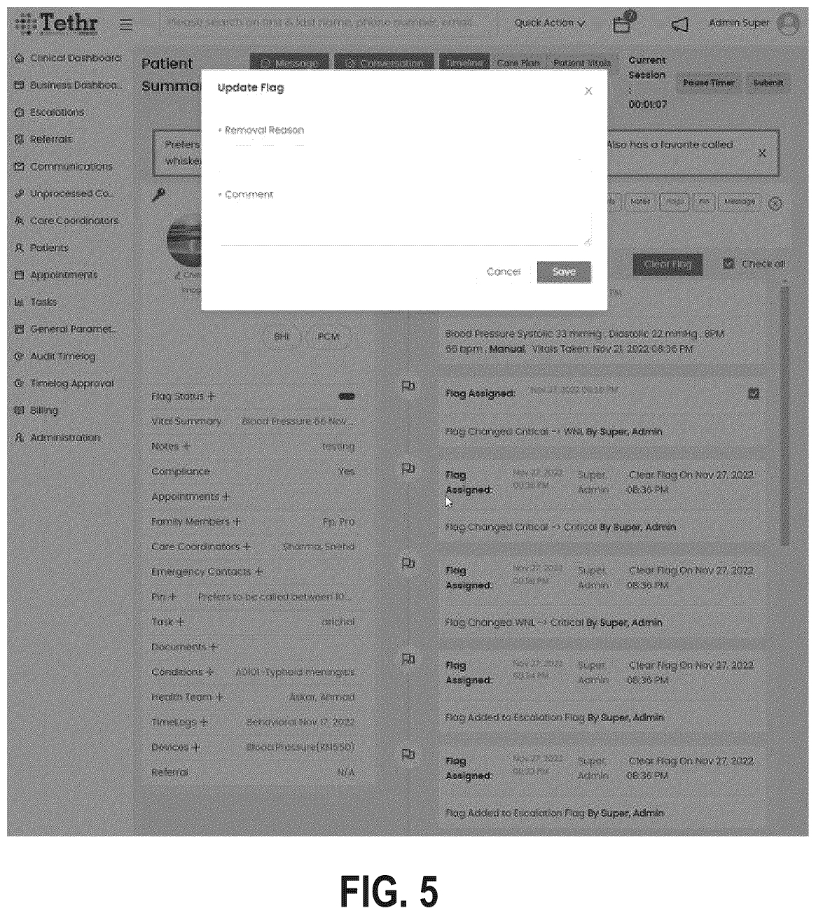

A user/administrator in the coordinated care system may require further details corresponding to a listed patient shown on FIG. 3 and may select a patient name from the list. FIG. 4 illustrates a patient summary view displayed once the first name on the list of FIG. 3 is selected. This view allows a user to have a complete picture of patient data in order to address various patient needs. On the left side, basic patient data is presented such as contact information, patient status, emergency contacts, heath care team members, medical devices in possession of the patient, etc. On the right side, a patient timeline has been generated by the system and is provided to the user (timeline view is also selected on the top buttons of FIG. 4). The patient timeline is a powerful tool that can list the patient's medical history and treatment details over the period known by the system (which is preferably the patient's entire history). The timeline may be sorted to show the most relevant data needed by an administrator. In the provided view of FIG. 4, the timeline is showing a sorted list of patient vitals readings information and relevant patient flags in timeline order. This view is provided based on selections of the selectable buttons located above the timeline. Other buttons could also be selected to provide additional information in the timeline (e.g., intake, appointments, device, documents, notes, pin, messages, health data, task). From the timeline, a user can click into an entry to view more information. Additionally, if there is a flag to address, the user can click the flag to open an additional entry screen (shown in FIG. 5) and either alter/provide information regarding the flag, clear the flag, and add notes about the flagged issue.

Additional options may be provided for with the patient detail screen of FIG. 4. For example, buttons on the top portion of the screen may be provided in order to facilitate communications with the patient or view previous communications. The additional Care Plan, Patient Vitals and Escalation buttons may also be provided (described below) in order to view different aspects of the patient data. A session timer may also be shown and controllable within a patient summary screen. Such a timer creates data that may be utilized by the system for billing purposes.

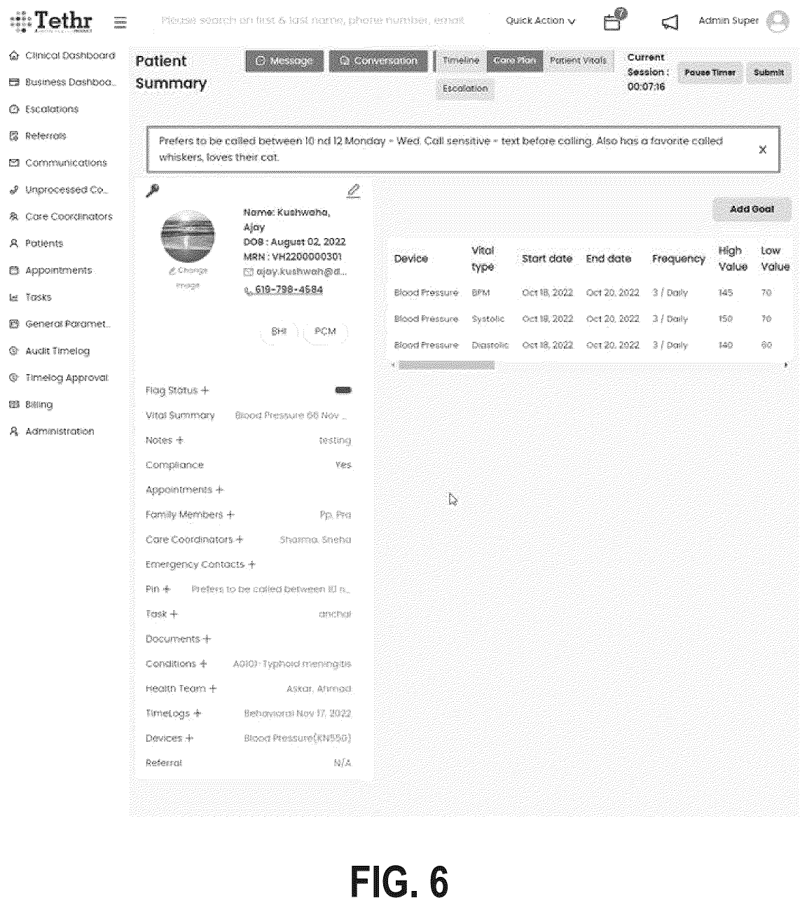

FIG. 6 shows an additional patient summary view when the Care Plan button is selected at the top portion of the screen. In this view, the timeline has been removed and patient care plan items are listed on the right side of the screen. In the current example, this patient's care plan includes taking specific vital readings of the patient three times daily. Additional items could be added either by an administrator or imported upon receiving provider data. Additional items may also be suggested by the system to the administrator to determine if such items should be added. Example additions to a care plan may include requirements for follow up appointments, medication instructions, and the like. This information may be utilized by an administrator of the coordinated care system to verify patient compliance and assist therewith, thereby tending to lead to improved patient outcomes.

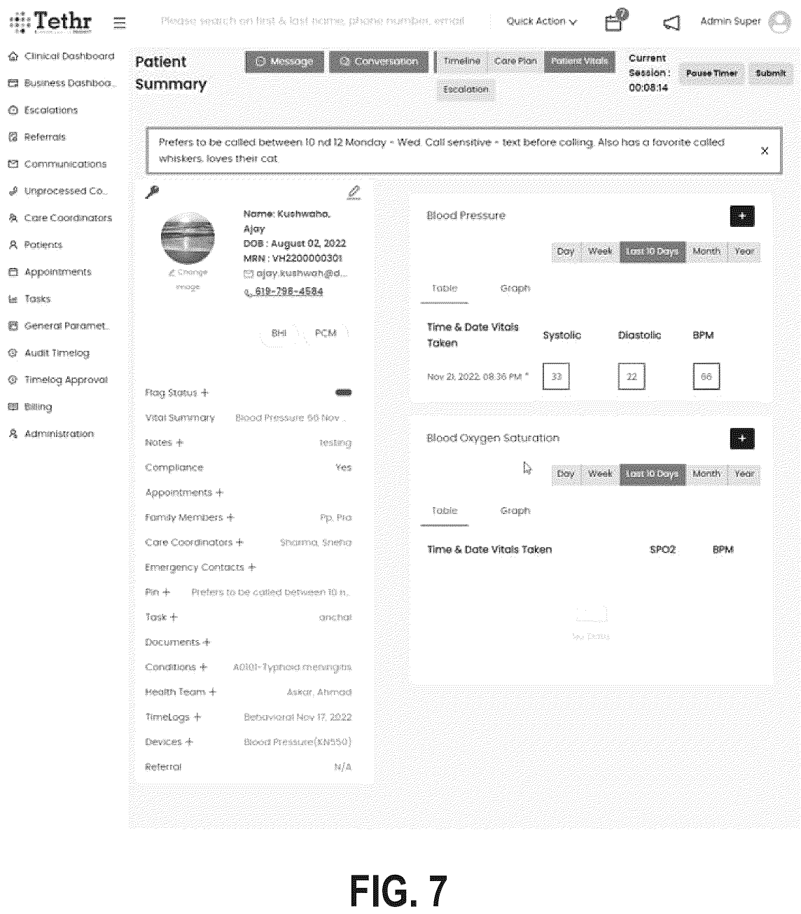

FIG. 7 illustrates an additional patient summary view when the Patient Vitals button is selected at the top portion of the screen. In this view, the timeline or care plan portion is removed and various patient vitals options are loaded on the right side of the screen. The present view shows blood pressure and oxygen saturation readings options. In these blocks, vital signs taken may be displayed. Such vitals may be received from, for example, manual entry from a user, provider data (e.g., taken from a physician/nurse encounter), from a user device (e.g., taken by a user and sent by a device), and/or from an external medical monitoring device in communication with the coordinated care system. The patient vitals view may also provide illustrative charts that show trends in the patient vitals over time.

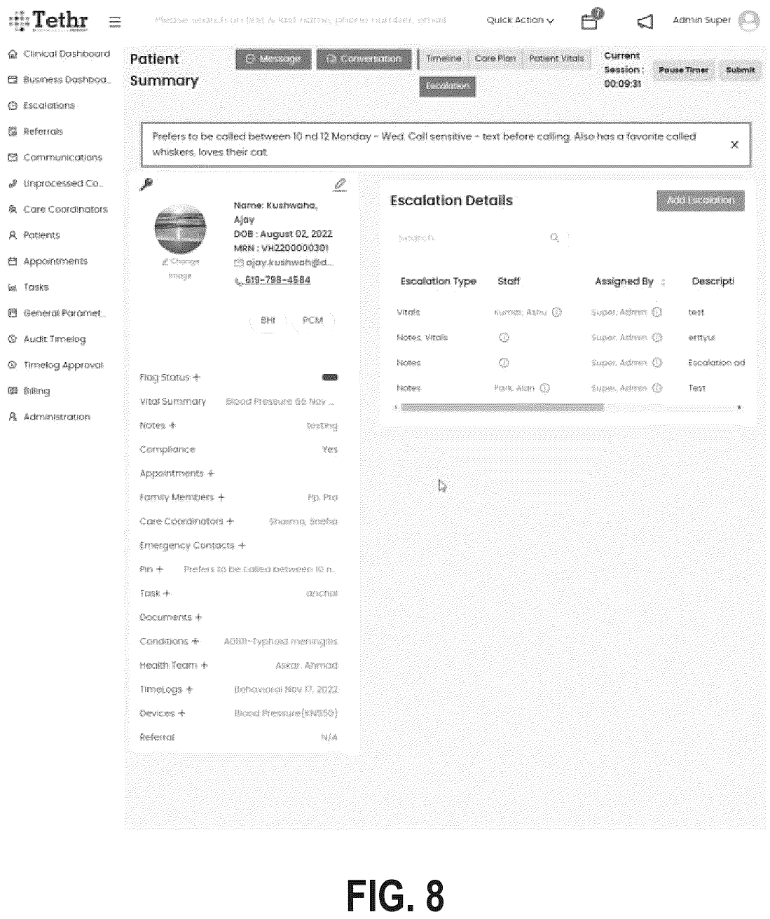

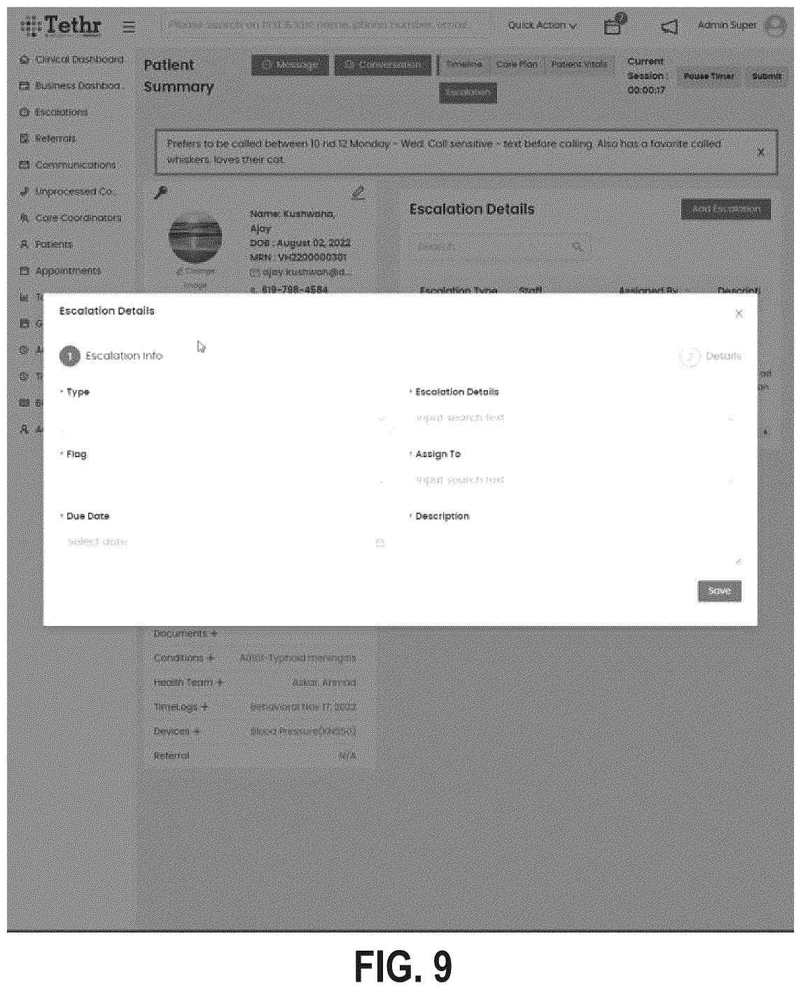

FIG. 8 illustrates an additional patient summary view when the Escalation button is selected at the top portion of the screen. In this view, the timeline, vitals or care plan portion is removed and various escalation details are loaded on the right side of the screen. The type and content of escalation priority entries will vary based on patient conditions/needs. Issues that may be flagged for escalation may be also received from, for example, manual entry from a user, provider data (e.g., taken from a physician/nurse encounter), from a user device (e.g., taken by a user and sent by a device), and/or from an external medical monitoring device in communication with the coordinated care system. The escalation view may also include an add escalation button that will load an escalation entry screen shown in FIG. 9. In this screen, a user may enter an escalation for a patient with corresponding details, priority assignment, flags, due dates, assign the escalation to an administrator to handle, etc. Once the details are entered, a new line will appear in the escalation list of FIG. 8. Additionally, corresponding flags and action items will appear on other relevant views of the coordinated care system.

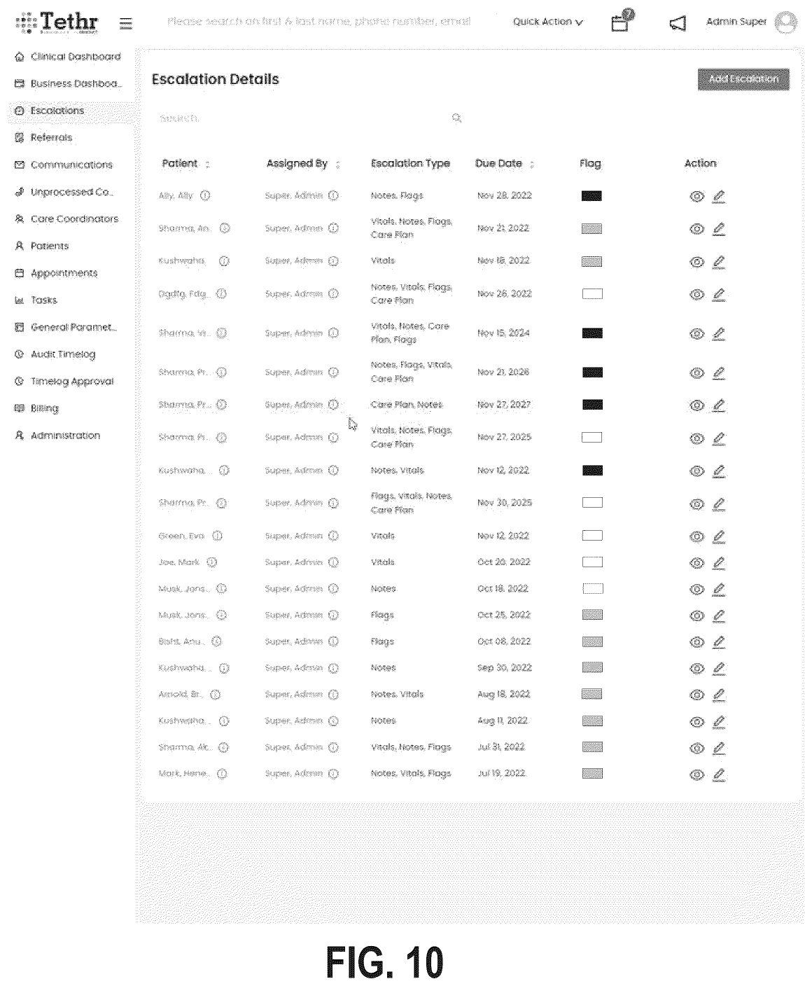

FIG. 10 illustrates a view of the coordinated care system when the escalation tab on the left side is selected. In this view, a patient list is provided with their corresponding escalations. The patients may be sorted by, for example, due date of the escalation. Alternatively, the patient list may be sorted by other methods such as by flag type, administrative assignment, etc. In this screen, an user may open the flag screen and edit the flag or add an escalation as shown above. Additionally, the user can enter an action taken with respect to the patient to document the patient's care.

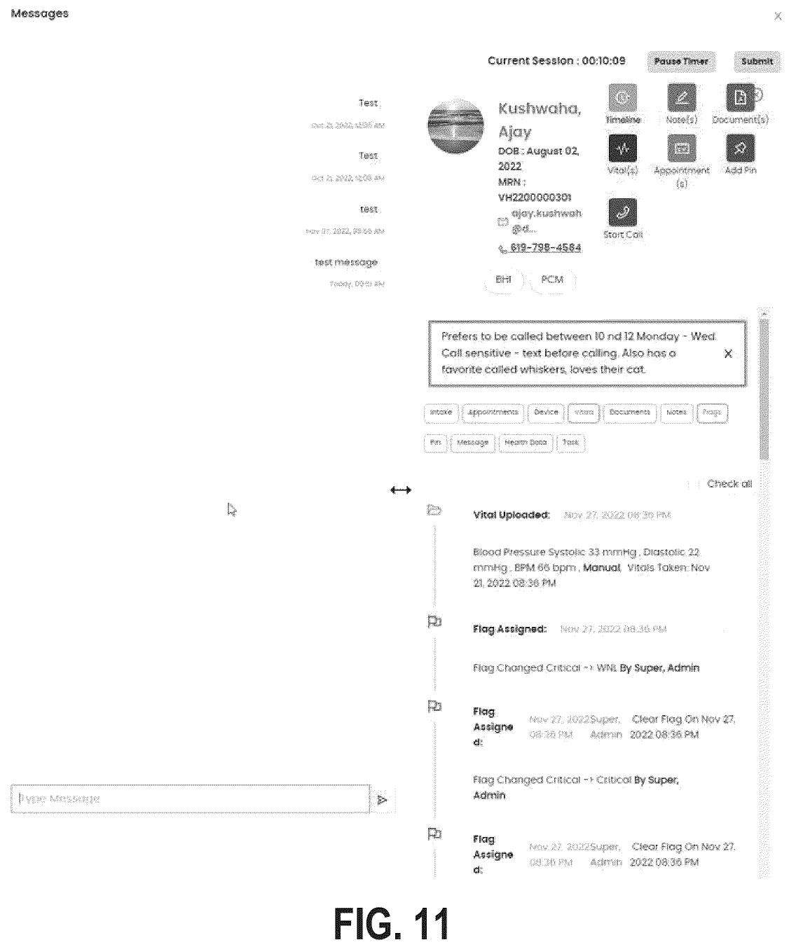

FIG. 11 illustrates a communication screen where an administrator can communicate with a patient in the coordinated care system. The communication screen may be utilized to implement a chat window. This chat may correspond to a chat window of an app executed on a device belonging to the patient. It may also show a view of an SMS conversation occurring between the patient and the system. The communication screen further includes contact details of the patient. Various patient data may also be readily accessible to the administrator of the system such as the timeline functionality described above, a schedule of appointments, vitals, other notes, and the like. Having such data readily available assists in having efficient interactions between patients and corresponding administrators providing care. Moreover, the communication screen may include the ability to initiate a call to the patient. A call may utilize the administrator's computer to call a patient's cell phone or land line. Alternatively, the call may connect to the patient via the patient app. The timer functionality shown in FIG. 3 may also be implemented across the screens shown above (and shown in FIG. 11) to continuously monitor time spent on a particular patient.

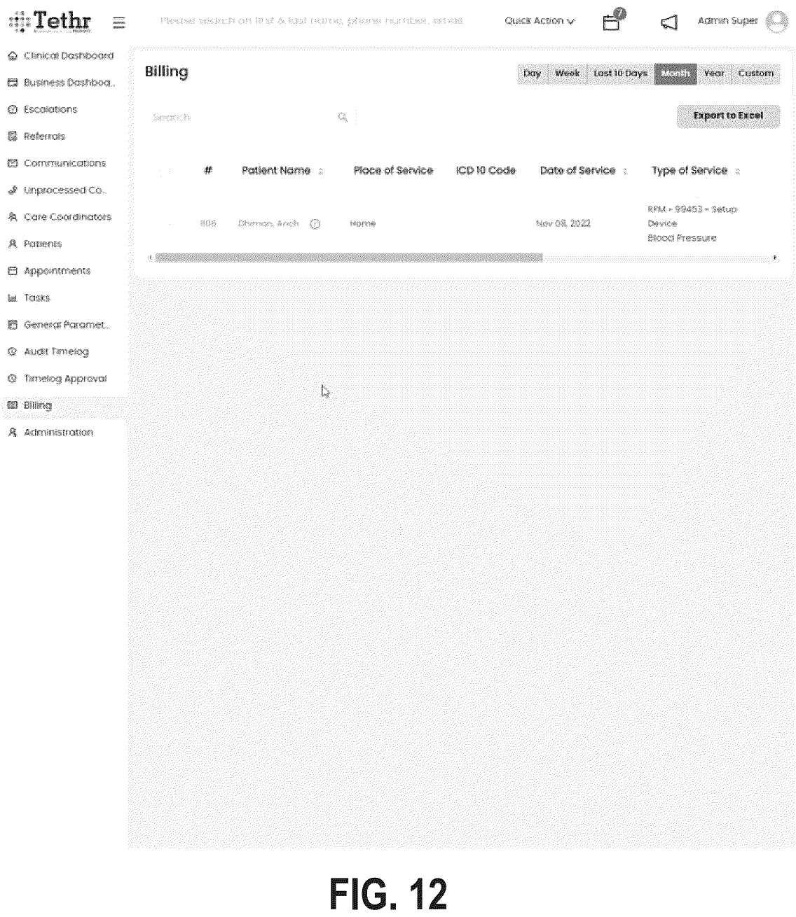

FIG. 12 illustrates a screen where the billing tab on the left side has been selected. It is appreciated that interactions that occur in the coordinated care system may be monitored and relevant data may be sent to a billing system. When a patient has had an interaction that can be billed, it will be allowed to show on the displayed list. Such bills may be transmitted to, e.g., an insurance company or a provider in the system that may bill for specific actions. The system may also provide a function to export the billing data to other systems in order to implement accounting practices.

It is appreciated that the patient information shown in the screenshots described above may originate from one or more providers, patients, third party records custodians, may be entered by internal users of the coordinated care system, and generally by a combination thereof. The data flow to the system may be implemented using a system, such as system 100. The aggregation of this data by, and the implementation of, the coordinated care system provides advantages over prior systems that are overly siloed and cannot provide complete pictures for patient care. Accordingly, the system not only provides technical efficiencies over prior art systems, it ultimately improves patient outcomes. It is further understood that the layouts and screens shown above are provided by way of example and numerous modifications may be implemented while still practicing aspects of the invention.

The compilation of data and organization described above provides patient care details in a more complete manner, which leads to the advantages discussed above and facilitates additional features. For example, according to one aspect, the system may generate a patient compliance score utilizing data corresponding to confirmation in the system that a patient has, for example, provided relevant vitals, taken appropriate medication, and the like. Tracking such patient compliance may help providers limit the occurrence of rehospitalization or further complications that may occur in the course of treatment for a patient. Patient compliance scores may also be utilized to motivate behavior of a patient. For example, a good compliance score may correspond to various discounts the insurer or provider may allow. In one aspect, such incentives may be automatically implemented within the billing system of the coordinated care system.

Further, a scoring system may be implemented in order to provide a rewards point system. In this instance, a patient may earn rewards for completing tasks administered by the system (e.g., for entering/providing vitals, verifying that medication has been taken, completing physician visits, etc.) and such rewards may be used, for example, in a rewards point store. In this manner, the coordinated care system along with the user app could be gamified to promote patient well-being.

It is further noted that as patients come into the coordinated care system a large amount of patient data will be ingested. In some aspects, such data may be utilized with AI or machine learning algorithms in order to predict patient needs, potential emergencies, alternative care action items, alternative diagnoses, and the like. For example, a machine learning algorithm implemented in the coordinated care system may monitor thousands of patients with similar conditions, taking similar medications, having similar vital data or patterns, etc. With this information, the machine learning algorithm may predict that a specific patient has differing needs or is at risk of a condition, e.g., a heart attack, based on trends in the patient's vital signs and in view of other data from a plurality of current and past patients. In this instance, the system may create an escalation that indicates to an administrator that the patient has a need, or even an urgent need, to take medication, provide more (and/or regular) data points, see a physician, to explore a different diagnosis or treatment course, and/or be more closely monitored by the system. Creating such a care suggestion may prevent larger catastrophic issues, while also saving time and money for all parties involved. Such a system may also be used to recognize that a patient coming into the system with one set of circumstances or treatment plan, may have different or other issues to address based on the AI/ML database knowledge. Similarly, the entrance of a patient into the system with a set of data may cause the system to reevaluate one or more existing patient's circumstances. The system may also take into account geographic, demographic and other circumstances which allows it to make more precise recognitions of circumstances in order to benefit patients in the system.

In one aspect, a coordinated care system may compile profile information for patients on the system. This information may be utilized to generate a patient profile that is usable in various aspects of the patient's care. For example, profile information may be compiled and utilized by the system to make determinations for care suggestions (e.g. to create action items for follow up, to make determinations for when additional help may be needed for patient compliance with medical advice, etc.) based on similarly situated patients with similar profiles. Moreover, profile information may be utilized to make determinations as described above with respect to AI/ML-based suggestions generated by the system.

In some aspects, system 100 may categorize patients and utilize psychographic profiles to assist the system to generate potential coordinated care action items, facilitate patient communications, and to more generally assist with maximizing patient compliance. For example, placing a patient in a particular psychographic profile may include profile assumptions as to how that patient is likely to be more responsive to feedback from the system. Such assumptions could include whether/how a patient responds to emails, text messages, app notifications, or phone calls. In some aspects, further utilization of AI or ML resources may monitor interactions with a patient and alter how the system interacts with the patient. For example, if a patient does not respond to various text messages, but does respond to email, the system may remove a type of communication to the patient. The system may also monitor the content of communications themselves to determine if there are better ways to convey information to ensure responsiveness. In such aspects, a generative AI system may also be utilized to generate communications that are more likely to be read and responded to by a patient.

In another aspect, psychographic data may be utilized to assist with patient interactions with a user of a system. For example, if a patient calls in to the system, the user of the system may have various data displayed to the user based on the patient's psychographic profile that will help to make the interaction more pleasant. E.g., if a patient calls in from a particular local area, the system may compile and display data about that area (such as weather, local sports scores, local news items, and the like) that may be used in conversation to help a patient be more comfortable and open in their care. Such features of system 100 provides for better experience management for a patient, which leads to improved compliance and ultimately improved patient outcomes.

Psychographic data may also be used to generate one or more psychographic scores. One example score may include a technology aptitude score. This score may indicate how well a patient can handle tasks that require different levels of technological skill. With this knowledge, the coordinated care system may indicate to a caregiver that using different medical equipment, different communication modes, etc., may be better suited to maximize positive patient outcomes. For example, using a simple medical device correctly may lead to better outcomes than using a more complicated device that is perhaps better if it means that a patient will be more compliant.

It is also noted that the above-discussed compilation of data allows for additional advantages. For example, in one aspect the coordinated care system may utilize inputs from both cellular phone/wearable device sensor data and other dedicated medical devices (such as a glucose monitor, CPAP machine, sleep monitoring systems and the like). Utilizing each of these types of devices over a multitude of patients allows for the system to model the more simple sensor data, e.g., phone sensor data, and therefore can allow for the cessation of use of the more specific and dedicated medical devices, (which can be more complicated to use and from which to retrieve readings).

The coordinated care system may also include functionality to auto generate coordinated care action items in response to data compiled and/or entered into the system. It may further generate forms to complete in order to meet these items. Such action items and/or corresponding forms may be for an administrator or a patient to complete. For example, if an escalation requires feedback from a patient or a reading from a patient device, the system may solicit and receive a reading of a patient device and/or generate a form and communicate that form to the patient. The system may cause the app to display a prompt for information or display the form in order to allow the patient to provide information or complete a form to provide the required information. This form may then be automatically sent back the system to complete the escalation, or provide more information to an administrator. Similarly, a coordinated care action item for an administrator may prompt the administrator to complete a task (e.g. follow up with a provider regarding one or more patient needs, feedback patient information to a provider, review patient compliance with one or more medical instructions such as taking medication and attending follow up appointments, arrange for ensuring patient compliance with instructions, etc.) and document completion. Such documentation may be implemented on a generated form.

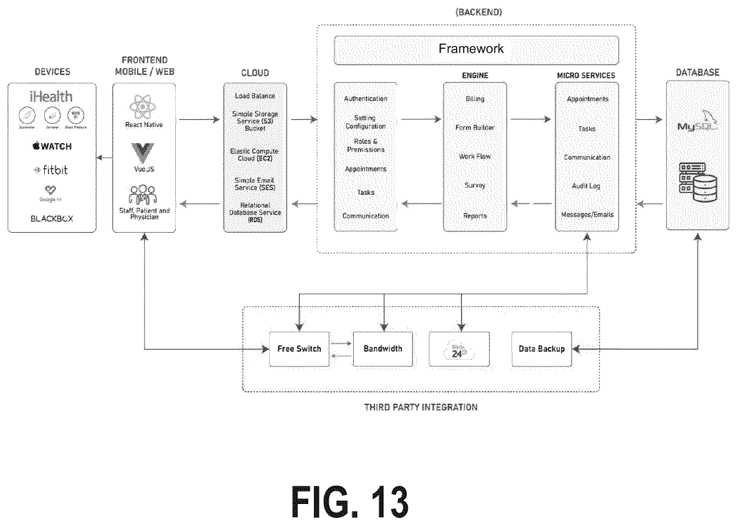

FIG. 13 illustrates an example system architecture according to an aspect of the present disclosure. A device, such as a patient device, may communicate patient data with a front end server corresponding to the coordinated care system. The front end server communicates with a back end service, which may be hosted on a cloud service (such as an Amazon® web services system). The back end system includes multiple functions to execute the systems described herein. The back end system may authenticate and monitor correspondence and allow data to be conveyed within the system. It may also be configured to administer tasks, communications, and other appointments in the system.

The back end system may further include an engine configured to administer specific modules. For example, a billing engine may be provided that monitors actions in the system in order to generate bills, request further information as needed, and to accept/distribute payments appropriately. The engine may also include a form builder that is able to generate various forms needed by users of the system. It is appreciated that the system will work with various types of providers which may require different information when onboarding a patient into the system, to accomplish billing tasks, etc. Providers may also want different information to be fed back from the system in order to adequately monitor their patients. For example, a physician that is treating a patient with the goal of managing pain may want different data (e.g., a pain score) than a physician handling a patient experiencing neurological issues (e.g., information relating to balance or weakness). Such specifics can be a entered into a form builder, which will then cause the system to obtain information as requested.

The engine may further include a workflow module. This module may create workflows geared to specific circumstances in order to better automate the system. For example, a workflow for monitoring a patient in transitional care may be different than a patient for chronic care, principal care, or remote patient monitoring. Workflows may also be different depending on how primary physicians are handling patients and by what those respective physicians want handled by the system. Some of these flows may be governed by what information billing codes require and what providers are required to do to meet a billing code. The workflow module may be further utilized to automatically create tasks and/or flags (e.g., an escalation task shown above) for an administrator of the coordinated care system to complete in order to fully realize a service for completion of a billing requirement. Accordingly, the workflow module may be configured to adapt to various rules of the coordinated care system based on one or more of different patients, different providers, different conditions being monitored, etc.

The back end further includes a microservices module. This module may be utilized to administer tasks such as setting up appointments in the system, arranging communications between parties, compiling logs and auditing data, and the like. The back end may be further in communication with the database to store data coming in and out of the system. Further, the system architecture may include third party integration systems which function to integrate the coordinated care system with respective systems of third parties such as providers, insurance billing entities, other technical services utilized by the system, and any other tool needed to facilitate integration of the backend processes with third parties.

The following examples illustrate data flow and capabilities of the coordinated patient care system in the context of use cases in handling patient care according to various aspects of the present disclosure. It is appreciated that these examples are not limiting and are provided for illustrative purposes only in order to enable a person of ordinary skill in the art to understand capabilities of the system. It is appreciated that in reading these examples a person of ordinary skill in the art would understand how communications/data flow are being handled in the coordinated care system and how these examples apply to the technological system of the aspects described above.

### EXAMPLE 1

Patient A was hospitalized and released into a skilled nursing facility. Upon entering the facility, Patient A is enrolled in the coordinated care system from one or more of a personal device, provider device, or onboarding kiosk as described above. Once Patient A's information is obtained, medical records are sent to the coordinated care application server. Such records will generally include diagnoses, discharge instructions, and the like. These records may be sent from one or more provider devices at the skilled nursing facility and/or the hospital. Records may likewise come from a separate records database. Additionally, back office processing actions may begin such as screening insurance, establishing billing plans, and the like. The coordinated care system may then function retrieve information as part of a care plan (e.g. have the patient respond to questions and/or report vitals through a user device; and obtain updated data from the skilled nursing facility) and may further generate/modify a care plan based on received data described in this example. The coordinated care system may further provide messages to patient A to help the patient further understand their healthcare needs. Such messages may include recommendations for questions that the patient may ask to a provider in order to be a better advocate for their own health care (e.g. questions regarding tests and/or medications that should be addressed). Such messages may be facilitated in-app via voice, text, email, direct message, and the like.

Upon discharge of Patient A from the skilled nursing facility, the coordinated care system will have the requisite data such that it may be utilized to assist Patient A to transition to the next phase of care. This may include working with the patient and coordinating medications, medical devices, social services, and the like. The system may further utilize data to anticipate needs of the patient. For example, in the event that the system is following up on the patient's mental health or medication compliance, the system may determine that Patient A is at risk for one or more adverse events. In response to this determination, one or more action items may be provided to an operator/user of the system to assist Patient A (such action items may also get various providers involved as appropriate). Such actions may prevent further adverse actions that will help patient outcomes, and may save the various providers substantial time and money as readmissions may be penalized or not be covered by various insurance providers.

### EXAMPLE 2

Patient B has a serious mental illness problem or substance abuse issues. This patient may come into the system from a state-run facility. In such circumstances, Patient B may come into the system after having interactions from multiple service providers over a period of time. These interactions may be disjointed or partially known to one another. Patient B may be enrolled into the system and his data transmitted to the coordinated care system from a records database from a service that compiles health information. This information may be complete, but also may have gaps. Where gaps exist, the coordinated care system may flag an operator to locate additional data or discuss with Patient B. It is appreciated that such a patient may have various needs for social services and may be receiving assistance from different services. The coordinated care system may further search the area of the patient and identify further services (whether local, State, Federal and/or private) that may assist Patient B and establish an action plan to obtain such assistance. In doing so, the coordinated care system further saves time/cost for these services after ensuring that Patient B's whole needs are met (which therefore reduces the likelihood of more costly adverse events).

### EXAMPLE 3

Patient C may be a patient that is struggling with physical or mental health, or perhaps on the verge of homelessness. Patient C comes into the coordinated care system on their own and registers themselves. Upon receiving Patient C's information, the coordinated care system may create an action/care plan to help patient C. For example, the system may connect Patient C to one or more providers and/or social services. In the event that Patient C does not want to seek assistance, the coordinated care system may still be able to assist Patient C with self-managing care (e.g., with respect to medications already in possession, to check vitals and other health care statistics, and the like). It is appreciated that otherwise healthy patients may utilize the system in a similar manner. In such a circumstance, the system may be utilized to track data over a period of time which will better allow the system to recognize potential issues using, e.g., predictive algorithms discussed above.

### EXAMPLE 4

Patient D is an older person who has lost her spouse and her children are busy taking care of their own children. Patient D is in the early stages of mild cognitive impairment, has minor mobility problems, and may be ready for assisted living. Patient D is having a difficult time remembering medications and should not be driving. Patient D's physician recommends that he/she utilize the coordinated care system and begins the process and provides Patient D's data upon enrollment. In such a circumstance, Patent D's physician understands that the patient is at risk for a serious issue (e.g., a fall), and does not have the time and resources to consistently follow up with Patient D due to other patient workload. Patient D's physician may provide a care plan to the coordinated care system. Additionally, the system may generate one by itself in light of the received data. Still further, the system may develop a hybrid plan based on physician recommendations and system recommendations.

Under a developed care plan, the system may follow up with Patient D regarding medications, implement wellness checks, and the like. Further, the system may utilize sensors on a patient device (e.g., phone) to retrieve vital information and activity information. Activity information may include sensing abnormal periods of inactivity that may be a cause for concern (e.g., due to lack of movement from Patient D or lack of interaction), likewise, sensors on the patient device may detect sudden falls or abnormal location data that may be of concern. The coordinated care system may be further linked to another party such as Patient D's child and provide one or more notifications regarding such potential issues relating to patient D. The system may further be utilized to put Patient D in contact with the linked party. The coordinated care system may further tie into other apps that can assist this patient such as a ride share program, grocery delivery, etc.

The systems described herein provide an immense improvement over the technological systems currently utilized in healthcare systems. Previously, a patient exiting a hospital is simply released and there is no system to ensure that that patient follows a physician's direction, and the hospitals merely hope that the patient does not return within 30 days. A physician handling chronic care may not necessarily know if one of their patients has a hospital visit and receives different instructions in response to a medical crisis. Additionally, physicians may not know if medications or other medical supplies are promptly and correctly delivered to a patient. Aspects of the present disclosure provide a new system capable of integrating with various siloed data points, and further capable of communicating more easily with a patient. Such a system not only provides technical efficiencies for parties involved in the care of patients, but it also generally provides better patient results.

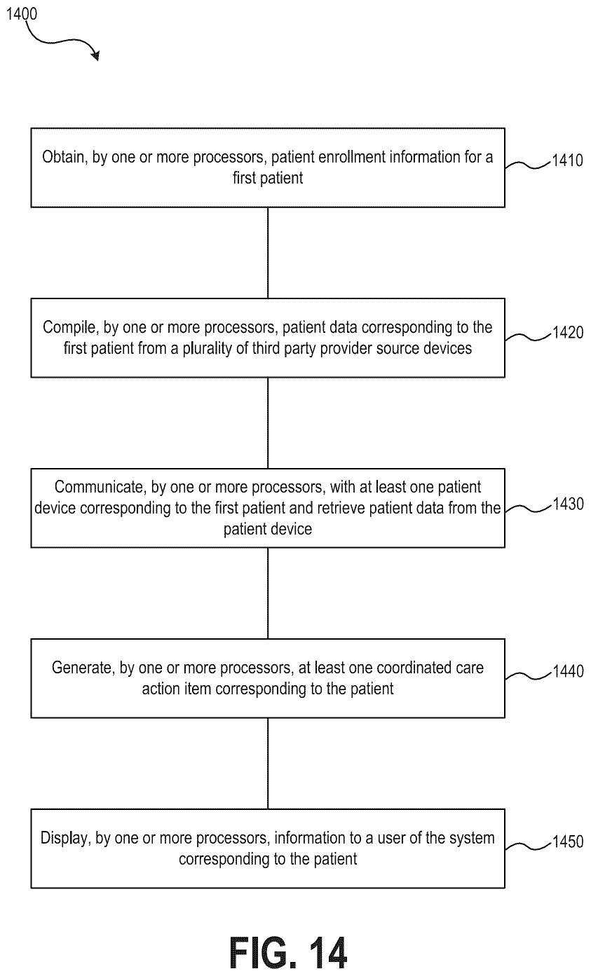

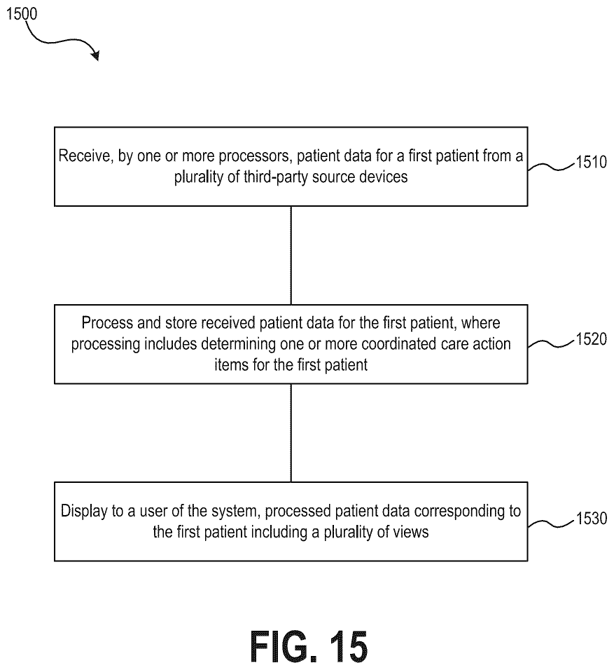

Referring to FIGS. 14-15, flow diagrams of an example methods according to one or more aspects of the present disclosure are shown as a methods 1400 and 1500 respectively. In some implementations, the operations of the methods 1400 and 1500 may be stored as instructions (e.g., the instructions 113 of FIG. 1) that, when executed by one or more processors (e.g., the one or more processors 111 of FIG. 1), cause the one or more processors to perform the operations of the methods 1400 and/or 1500. In some implementations, the methods 1400 and 1500 may be performed by a computing device, such as computing device 110 of FIG. 1 and/or in combination with other devices of FIG. 1 as appropriate.

At step 1410, method 1400 includes obtaining, by one or more processors, patient enrollment information for a first patient. At step 1420, method 1400 includes compiling, by one or more processors, patient data corresponding to the first patient from a plurality of third party provider source devices. Such patient data may include data from a computing system administered by one or more of a hospital, physician's office, pharmacy, and a medical device provider. Patient data may also manually entered at a device corresponding to an administrator of a coordinated care system. At step 1430, method 1400 includes communicating, by one or more processors, with at least one patient device corresponding to the first patient and retrieving patient data from the patient device.

In response to one or more of the compiled patient data and patient data retrieved from the patient device, method 1400 includes at step 1440, generating by one or more processors at least one coordinated care action item corresponding to the patient. In some aspects, a coordinated care action item may include an action to be displayed to the user of the system to cause the user to follow up with the patient regarding a care compliance item. Such an item may also include an item displayed to the user of the system to follow up with one or more providers for a patient in order to facilitate a patient care task. Moreover, a coordinated care action item may be generated from a predictive model based on a dataset corresponding to a plurality of patients.

At step 1450, method 1400 includes displaying, by one or more processors, information to a user of the system corresponding to the patient. In some aspects, such a display may include various displays as discussed above in FIGS. 2-12 and may include, for example, a display of information corresponding to a timeline view for the first patient. This view may include patient medical history, care history, and may also include one or more escalation items for the patient.

At step 1450, method 1400 includes receiving, by one or more processors, input data related to at least one coordinated care action item. Such input data may be received from a patient mobile device and/or a medical device corresponding to the patient. Such data may include health data and may include data related to a coordinated care action item. At step 1460, method 1400 includes updating and storing, by one or more processors, data corresponding to the first patient in response to the received input data.

Referring to FIG. 15, method 1500 is an example method to implement a coordinated care system configured to gather data from third party sources and to generate a display interface to assist a user managing care for a plurality of patients. Method 1500 may include at step 1510 receiving, by one or more processors, patient data for a first patient from a plurality of third-party source devices. At step 1520, method 1500 includes processing and storing said received patient data for the first patient, where processing includes determining one or more coordinated care action items for the first patient. Further, at step 1530, method 1500 includes the step of displaying to a user of the system, processed patient data corresponding to the first patient including a plurality of views. These views include at least one view includes a timeline view configured to display a plurality of data items (including at least one coordinated care action item) for the first patient received from the plurality of devices in timeline order. The timeline view may be further configured to selectively display data item types based on a selection of a user.

According to more detailed aspects of method 1500, a plurality of selectable buttons may be displayed which allow for the selection and deselection of types of data items of the plurality of data items to display. The selectable buttons may include one or more of flags, vitals appointments, devices, documents, notes, pin, messages, health data, tasks, and the like. In another aspect, method 1500 may display one or more of a care plan view which illustrates action items and entries corresponding to a patient care plan, an escalation view to a user that highlights escalation action items for the first patient, or a patient vitals view which illustrates historic vital readings for a selected patient.

In yet another aspect of method 1500 the one or more processors may generate one or more predictions for future care action items of a patient based on received patient data from the plurality of third-party sources and from the one or more patient devices. Such predictions may be displayed in the timeline view. One or more predictions for future care action items of the first patient may also be generated based on a data model utilizing received patient data corresponding to a plurality of patients.

It is noted that other types of devices and functionality may be provided according to aspects of the present disclosure and discussion of specific devices and functionality herein have been provided for purposes of illustration, rather than by way of limitation. It is noted that the operations of the methods and systems described may be performed in any order, or that operations of one method may be performed during performance of another method.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Components, the functional blocks, and the modules described herein with respect to the Figures) include processors, electronics devices, hardware devices, electronics components, logical circuits, memories, software codes, firmware codes, among other examples, or any combination thereof. In addition, features discussed herein may be implemented via specialized processor circuitry, via executable instructions, or combinations thereof.

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Skilled artisans will also readily recognize that the order or combination of components, methods, or interactions that are described herein are merely examples and that the components, methods, or interactions of the various aspects of the present disclosure may be combined or performed in ways other than those illustrated and described herein.

The various illustrative logics, logical blocks, modules, circuits, and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules, and circuits described in connection with the aspects disclosed herein may be implemented or performed with a general purpose single-or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or any conventional processor, controller, microcontroller, or state machine. In some implementations, a processor may also be implemented as a combination of computing devices, such as a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or any combination thereof. Implementations of the subject matter described in this specification also may be implemented as one or more computer programs, that is one or more modules of computer program instructions, encoded on a computer storage media for execution by, or to control the operation of, data processing apparatus.

If implemented in software, the functions may be stored on or transmitted over as one or more instructions or code on a computer-readable medium. The processes of a method or algorithm disclosed herein may be implemented in a processor-executable software module which may reside on a computer-readable medium. Computer-readable media includes both computer storage media and communication media including any medium that may be enabled to transfer a computer program from one place to another. A storage media may be any available media that may be accessed by a computer. By way of example, and not limitation, such computer-readable media can include random-access memory (RAM), read-only memory (ROM), electrically erasable programmable read-only memory (EEPROM), CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, hard disk, solid state disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to some other implementations without departing from the spirit or scope of this disclosure. Thus, the claims are not intended to be limited to the implementations shown herein, but are to be accorded the widest scope consistent with this disclosure, the principles and the novel features disclosed herein.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted may be incorporated in the example processes that are schematically illustrated. For example, one or more additional operations may be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, some other implementations are within the scope of the following claims. In some cases, the actions recited in the claims may be performed in a different order and still achieve desirable results.

As used herein, including in the claims, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. the term “or,” when used in a list of two or more items, means that any one of the listed items may be employed by itself, or any combination of two or more of the listed items may be employed. For example, if a composition is described as containing components A, B, or C, the composition may contain A alone; B alone; C alone; A and B in combination; A and C in combination; B and C in combination; or A, B, and C in combination. Also, as used herein, including in the claims, “or” as used in a list of items prefaced by “at least one of” indicates a disjunctive list such that, for example, a list of “at least one of A, B, or C” means A or B or C or AB or AC or BC or ABC (that is A and B and C) or any of these in any combination thereof. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed aspect, the term “substantially” may be substituted with “within [a percentage] of” what is specified, where the percentage includes 0.1, 1, 5, and 10 percent; and the term “approximately” may be substituted with “within 10 percent of” what is specified. The phrase “and/or” means and or.

Although the aspects of the present disclosure and their advantages have been described in detail, it should be understood that various changes, substitutions and alterations can be made herein without departing from the spirit of the disclosure as defined by the appended claims. Moreover, the scope of the present application is not intended to be limited to the particular implementations of the process, machine, manufacture, composition of matter, means, methods and processes described in the specification. As one of ordinary skill in the art will readily appreciate from the present disclosure, processes, machines, manufacture, compositions of matter, means, methods, or operations, presently existing or later to be developed that perform substantially the same function or achieve substantially the same result as the corresponding aspects described herein may be utilized according to the present disclosure. Accordingly, the appended claims are intended to include within their scope such processes, machines, manufacture, compositions of matter, means, methods, or operations.

What is claimed is:

1. A coordinated care system configured to gather data from third party sources and to generate a display interface to assist a user managing care for a plurality of patients, said system comprising:
a memory device for storing patient data records, where data corresponding to a respective patient is compiled from a plurality of third party sources; and
one or more processors communicatively coupled to the memory device, the one or more processors configured to:
receive patient data for a first patient from a plurality of third party source devices;
process and store said received patient data for the first patient, wherein said processing includes determining one or more coordinated care action items for the first patient;
display, to the user of the system, processed patient data corresponding to the first patient, said display including a plurality of views, wherein said display includes generating at least one view includes a timeline view configured to display a plurality of data items corresponding to data received for the first patient from the plurality of third party sources in timeline order, said plurality of data items including at least one determined coordinated care action item, wherein the timeline view is further configured to selectively display data item types based on a selection of a user.

2. The system of claim 1 wherein the one or more processors are further configured to display a plurality of selectable buttons which allow for the selection and deselection of data items of the plurality of data items to display.

3. The system of claim 2 wherein the selectable buttons include one or more of flags, vitals appointments, devices, documents, notes, pin, messages, health data, tasks.

4. The system of claim 1 wherein the one or more processors are further configured to display a care plan view which illustrates action items and entries corresponding to a patient care plan.

5. The system of claim 1 wherein the one or more processors are further configured to display an escalation view to a user that highlights escalation action items for the first patient.

6. The system of claim 1 wherein the one or more processors are further configured to display a patient vitals view which illustrates historic vital readings for a selected patient.

7. The system of claim 1 wherein the one or more processors are further configured to receive patient data for the first patient from one or more patient devices.

8. The system of claim 7 wherein the one or more processors are further configured to generate one or more predictions for future care action items of a patient based on received patient data from the plurality of third-party sources and from the one or more patient devices.

9. The system of claim 8 wherein said one or more predictions are displayed in the timeline view.

10. The system of claim 1 wherein the one or more processors configured to generate one or more predictions for future care action items of the first patient based on a data model utilizing received patient data corresponding to a plurality of patients.

11. A method for implementing a coordinated care system configured to gather data from third party sources and to generate a display interface to assist a user managing care for a plurality of patients, said method comprising:
receiving, by one or more processors, patient data for a first patient from a plurality of third party source devices;
processing and storing said received patient data for the first patient, wherein said processing includes determining one or more coordinated care action items for the first patient;
displaying, to the user of the system, processed patient data corresponding to the first patient, said display including a plurality of views, wherein said display includes generating at least one view includes a timeline view configured to display a plurality of data items corresponding to data received for the first patient from the plurality of third party sources in timeline order, said plurality of data items including at least one determined coordinated care action item, wherein the timeline view is further configured to selectively display data item types based on a selection of a user.

12. The method of claim 11 further comprising displaying a plurality of selectable buttons which allow for the selection and deselection of data items of the plurality of data items to display.

13. The method of claim 12 wherein the selectable buttons include one or more of flags, vitals appointments, devices, documents, notes, pin, messages, health data, tasks.

14. The method of claim 11 further comprising displaying a care plan view which illustrates action items and entries corresponding to a patient care plan.

15. The method of claim 11 further comprising displaying an escalation view to a user that highlights escalation action items for the first patient.

16. The method of claim 11 further comprising displaying a patient vitals view which illustrates historic vital readings for a selected patient.

17. The method of claim 11 wherein the one or more processors are further configured to receive patient data for the first patient from one or more patient devices.

18. The method of claim 17 further comprising generating, by one or more processors, one or more predictions for future care action items of a patient based on received patient data from the plurality of third-party sources and from the one or more patient devices.

19. The method of claim 18 wherein said one or more predictions are displayed in the timeline view.

20. The method of claim 11 further comprising generating, by one or more processors, one or more predictions for future care action items of the first patient based on a data model utilizing received patient data corresponding to a plurality of patients.
